(19) United States
(12) Patent Application Publication
(10) Pub. No.: US 2016/0022045 A1
(43) Pub. Date: Jan. 28, 2016

(54) Travel Eye Mask With Head Support Cushions

(71) Applicant: Zinus Inc., San Leandro, CA (US)

(72) Inventor: Dong Young Lee, Busan (KR)

(21) Appl. No.: 14/558,974

(22) Filed: Dec. 3, 2014

(30) Foreign Application Priority Data
Jul. 25, 2014 (CN) 201420414184.1

(51) Int. Cl. A47C 7/38; D05B 93/00; D05B 11/00; D05B 1/00; A61F 9/04; B60N 2/48

(52) U.S. Cl. CPC A47C 7/383; A61F 9/04; B60N 2/4876; D05B 11/00; D05B 1/00; D05B 93/00

(57) ABSTRACT

A travel eye mask assembly includes an eye mask connected by elastic straps to a headrest cushion. The headrest cushion includes a first cushion portion, a second cushion portion, and a less cushioned middle portion. The second cushion portion is connected to the first cushion portion through the middle portion. The eye mask includes a left eye patch and a right eye patch. A first strap connects the first cushion portion to the left eye patch, and a second strap connects the second cushion portion to the right eye patch. The first cushion portion is foldable over the second cushion portion when the left eye patch is folded into the right eye patch and the eye mask is disposed between the first and second cushion portions. An inner compartment that contains the eye mask is formed between the first and second cushion portions when the headrest cushion is zipped closed.

### CROSS REFERENCE TO RELATED APPLICATION

This application is based on and hereby claims the benefit under 35 U.S.C. §119 from Chinese Patent Application No. 201420414184.1, filed on Jul. 25, 2014, in China, the contents of which are hereby incorporated by reference. This application is a continuation-in-part of Chinese Application No. 201420414184.1.

### TECHNICAL FIELD

The present invention relates generally to eye masks and, more particularly, to a travel eye mask with an attached supportive headrest cushion that permits a more restful and comfortable travel experience.

### BACKGROUND

Airplane passengers typically attempt to sleep or at least rest at some point during the flight. Falling asleep can be difficult, however, amidst the commotion, noise and lights of an airplane cabin. To help business class passengers to fall asleep, airlines typically provide complimentary eye shades to block out cabin light. However, eye shades are not complimentary in economy class. Moreover, airline eye shades are typically thin and too small for many passengers so that light still enters around the curled corners.

Falling asleep in an airplane seat can also be assisted by supporting the passenger's head so that the passenger's neck need not hold the head upright against the seatback while sleeping. Providing external head support allows the passenger's neck muscles to relax and prevents neck aches caused by sleeping with a slouching neck. Head support can be provided by C-shaped cushions that wrap around the passenger's neck. However, these C-shaped cushions are hardly ever complimentary, and most passengers do not bring their own because the cushions are bulky. Many airline seats now include side flaps in the headrests that can be folded forward to support the passenger's head while resting. The distance between the flaps is so large, however, that practically no one's head would be cradled between both flaps. So the head of the passenger rests to the side against one or the other of the side flaps.

A device is sought for assisting a passenger to rest in an airplane seat that both provides head support and effectively blocks out cabin light without being bulky.

### SUMMARY

An eye cover device combines an eye mask with a headrest cushion. The headrest cushion and attached eye mask increase the user's comfort during extended travel in a slightly reclined airplane seat. The headrest cushion is positioned between the back of a user's head and the seatback to provide upright support for the user's head. The eye mask is connected to the headrest cushion by elastic straps. The eye mask provides a blind for darkened sleeping, and the headrest cushion supports the back of the user's head on both sides of the upper neck. The elastic straps and the ends of the headrest cushion cover most of the user's ears and block out some of the cabin noise.

The headrest cushion includes a first cushion portion, a second cushion portion and a less cushioned middle portion. The user's upper neck is supported between the first cushion portion and the second cushion portion. Because the middle portion of the headrest cushion is less cushioned and thinner than the outer cushion portions, the user's head falls farther back into the middle of the headrest cushion and is cradled between the two outer cushion portions. Thus, the user's head is prevented from moving to the right or left, and the user's neck does not bend or turn. Supporting the user's head from both sides holds the head upright against the headrest of the airplane seat and allows the user to relax his neck muscles, making it easier to sleep.

In another embodiment, a travel eye mask assembly includes an eye mask that is connected by elastic straps to a headrest cushion. The headrest cushion includes a first cushion portion, a second cushion portion, and a less cushioned middle portion. The first and second cushion portions are thinner near the middle portion than away from the middle portion. The second cushion portion is connected to the first cushion portion through the middle portion. The eye mask includes a left eye patch portion and a right eye patch portion. A first strap connects the first cushion portion to the left eye patch portion, and a second strap connects the second cushion portion to the right eye patch portion. The combined length of the first cushion portion, the middle portion and the second cushion portion is between ten and twelve inches, and the headrest cushion is sized to cradle an adult person's head at the upper neck.

The first cushion portion is foldable over the second cushion portion when the left eye patch portion is folded into the right eye patch portion and the eye mask is disposed between the first cushion portion and the second cushion portion. An inner compartment that contains the eye mask is formed between the first cushion portion and the second cushion portion when the headrest cushion is zipped closed.

A method of making a travel eye cover device includes sewing seams, folding portions of the device and zipping the folded device closed. A first seam is sewn in a headrest cushion to separate a first cushion portion from the remainder of the headrest cushion. A second seam is then sewn in the headrest cushion to separate a second cushion portion from the remainder of the headrest cushion. A middle portion of the headrest cushion is formed between the first and second seams. Before the seams are sewn, the majority of the padding material in the headrest cushion is pulled towards the ends so that less cushioning material is present in the middle portion and so that the first and second cushion portions are thicker towards the ends. A first zipper track is then attached to the first cushion portion, and a second zipper track is attached to the second cushion portion. The fabric strip of each zipper track is sewn to a fabric covering flap on each of the first and second cushion portions. A left eye patch of an eye mask is attached to the first cushion portion, and a right eye patch of the eye mask is attached to the second cushion portion. The eye patches are attached to the cushion portions by elastic straps.

The travel eye cover device is then packaged by folding the left eye patch over the right eye patch. The folded eye mask is placed between the first cushion portion and the second cushion portion that are folded over one another. Then the first cushion portion is zipped to the second cushion portion by zipping the first zipper track to the second zipper track. Closing the zipper fastens a first covering flap of the first cushion portion to a second covering flap of the second cushion portion and forms an inner compartment in which the eye mask is stored. The travel eye cover device is in a condition ready for delivery to the user after the zipper has been closed.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 is a perspective view of an eye mask and headrest cushion assembly that helps passengers to sleep while traveling in an upright, seated position for extended periods.

FIG. 2 shows the travel eye mask assembly of FIG. 1 as a left eye patch is partially folded into a right eye patch.

FIG. 3 shows the eye mask assembly of FIG. 1 with the crease between the folded left and right eye patches placed over a middle portion of the headrest cushion.

FIG. 4 shows the eye mask assembly of FIG. 1 folded and zipped closed in a compact state.

FIG. 5 is a front view of the eye mask assembly of FIG. 1 in which the eye mask has been removed from the inner compartment between the cushion portions.

FIG. 6 shows the eye mask assembly of FIG. 1 placed over the head of a passenger sitting in a slightly reclined airplane seat.

FIG. 7 is a flowchart of steps for manufacturing the travel eye mask assembly of FIG. 1.

### DETAILED DESCRIPTION

Reference will now be made in detail to some embodiments of the invention, examples of which are illustrated in the accompanying drawings.

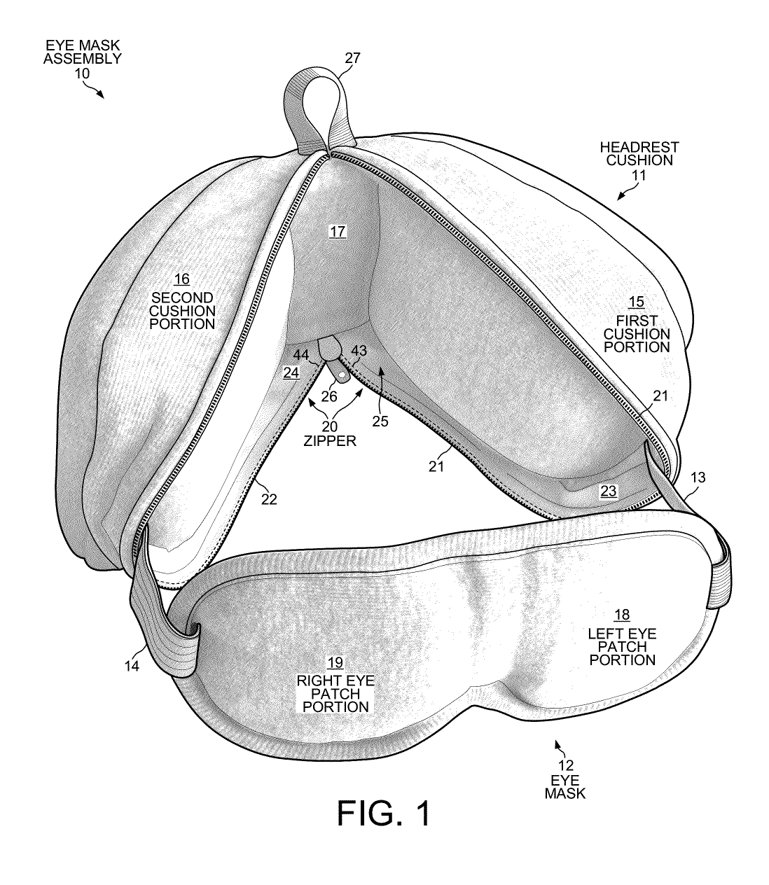

FIG. 1 is a perspective view of an eye mask and headrest cushion assembly 10 that helps passengers to rest while traveling in an airplane, train or bus seat. The travel eye mask assembly 10 both provides head support and blocks out light. Moreover, the eye mask assembly 10 folds into a small, compact unit that can be easily stored in carry-on luggage. The travel eye mask assembly in FIG. 1 is zipped open and ready for use.

The eye mask assembly 10 includes a headrest cushion 11 that is connected to an eye mask 12 by elastic straps 13-14. The headrest cushion 11 includes a first cushion portion 15, a second cushion portion 16 and a middle portion 17. The eye mask 12 has a left eye patch portion 18 and a right eye patch portion 19. The first strap 13 connects the first cushion portion 15 to the left eye patch portion 18, and the second strap 14 connects the second cushion portion 16 to the right eye patch portion 19.

The headrest cushion also has a zipper 20. The zipper 20 includes a first zipper track 21 and a second zipper track 22. The first zipper track 21 is attached to the first cushion portion 15, and the second zipper track 22 is attached to the second cushion portion 16. Each of the cushion portions 15-16 has a fabric covering flap 23-24. Each of the zipper tracks 21-22 is sewn onto the edge of a covering flap 23-24. An inner compartment 25 is formed between the first cushion portion 15 and the second cushion portion 16 when the cushion portions are folded towards each other and the covering flaps are zipped into each other by the zipper 20. FIG. 1 shows the zipper tab 26 that is used to close and open the zipper 20.

One end of each strap 13-14 is sewn into an edging seam that surrounds the eye mask 12. The other end of each strap 13-14 is attached to a cushion portion 15-16 inside the inner compartment 25. This allows the compartment 25 to be zipped closed after the eye mask 12 is folded and inserted into the compartment 25. The left eye patch portion 18 is folded into the right eye patch portion 19, and the folded eye mask 12 is placed between the first cushion portion 15 and the second cushion portion 16 as the cushion portions are folded over one another. When the cushion portions 15-16 are folded and zipped closed, the eye mask 12 fits between the cushion portions 15-16. FIG. 1 also shows a loop 27 that can be used as a handle to carry the eye mask assembly 10 in the compact, closed state.

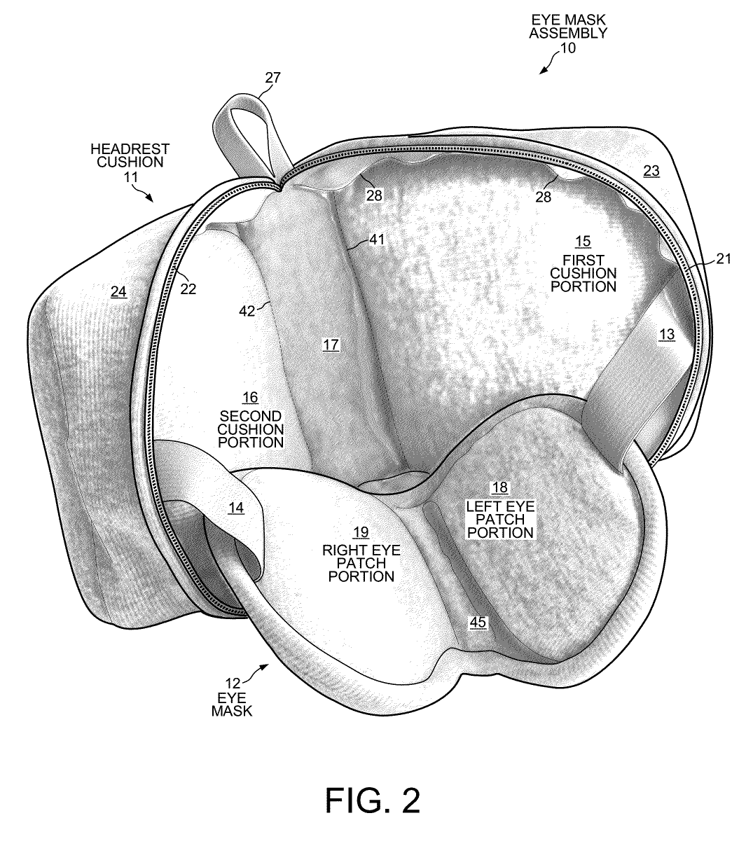

FIG. 2 shows the travel eye mask assembly 10 as the left eye patch portion 18 is partially folded into the right eye patch portion 19. The crease between the left and right eye patch portions 18-19 is then moved towards the middle portion 17 of the headrest cushion 11 before the assembly is closed. FIG. 2 also shows the wavy loose side 28 of the fabric strips of the zipper tracks 21-22. The seam that attaches the zipper tracks 21-22 to the edge of the covering flaps 23-24 is sewn closer to the zipper teeth than to the loose side 28.

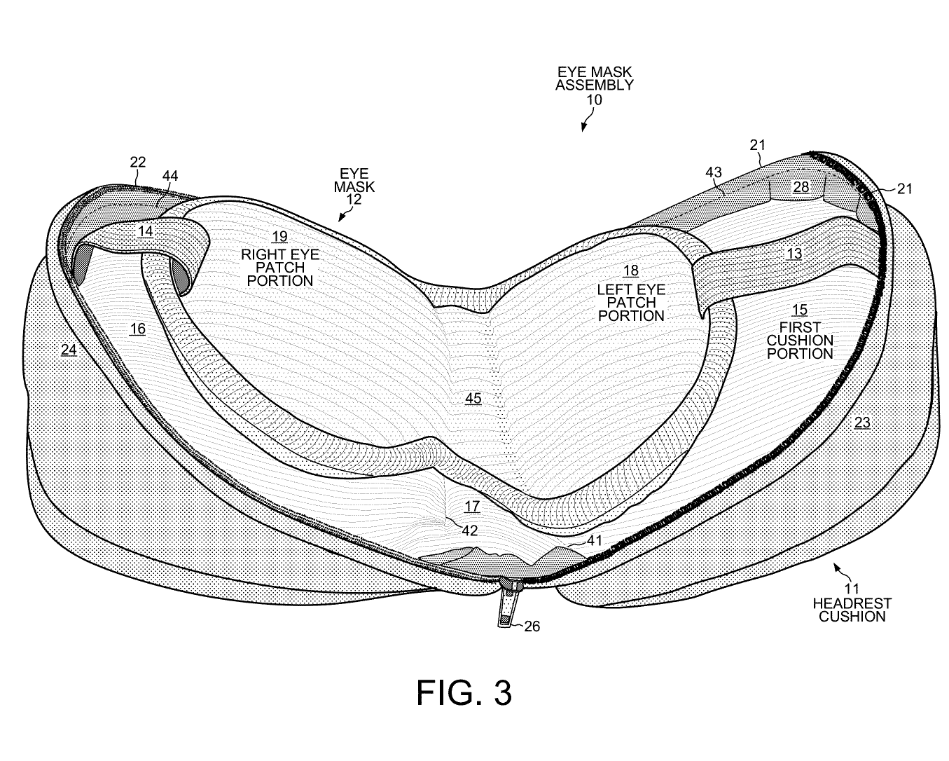

FIG. 3 shows the travel eye mask assembly 10 with the crease between the left and right eye patch portions 18-19 placed over the middle portion 17 of headrest cushion 11. The assembly 10 can now be closed by folding the first cushion portion 15 over the second cushion portion 16 as the left eye patch portion 18 is folded into the right eye patch portion 19. FIG. 3 shows that both the first and second cushion portions 15-16 are thinner near the middle portion 17 than away from the middle portion. The middle portion 17 is less cushioned and therefore thinner than the cushion portions 15-16. Thus, more foam or padding material is located at the ends of the headrest cushion 11 than in the middle. When a user places the headrest cushion 11 behind his head and leans back into the headrest of a seat, the thicker ends of the headrest cushion 11 tend to cradle the user's head between them. The cushion portions 15-16 support the user's head from both sides and hold the head upright on the headrest. The first cushion portion 15, the middle portion 17 and the second cushion portion 16 have a combined length of less than twelve inches. Therefore, the user's head does not rest on either one or the other cushion portion as occurs with the side flaps on airline seat headrests. Instead, both sides of the user's upper neck are supported at the same time by the two cushion portions 15-16.

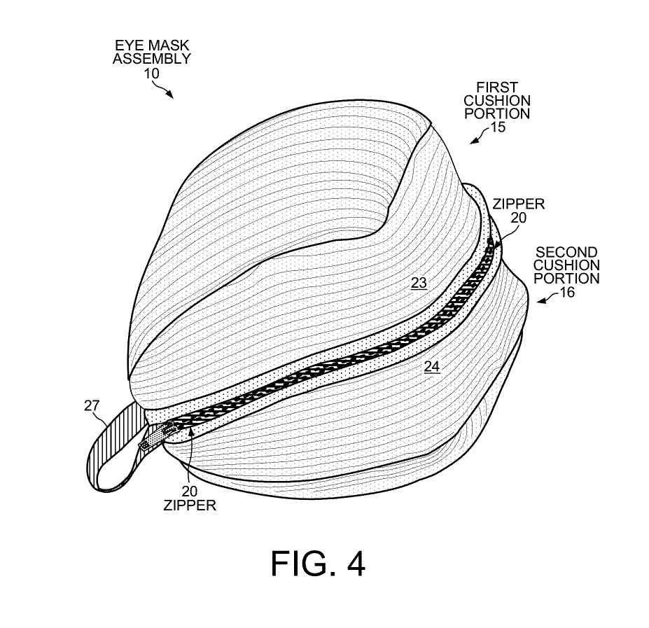

FIG. 4 shows the eye mask assembly 10 folded and zipped closed in a compact state. The eye mask 12 is disposed between the first cushion portion 15 and the second cushion portion 16.

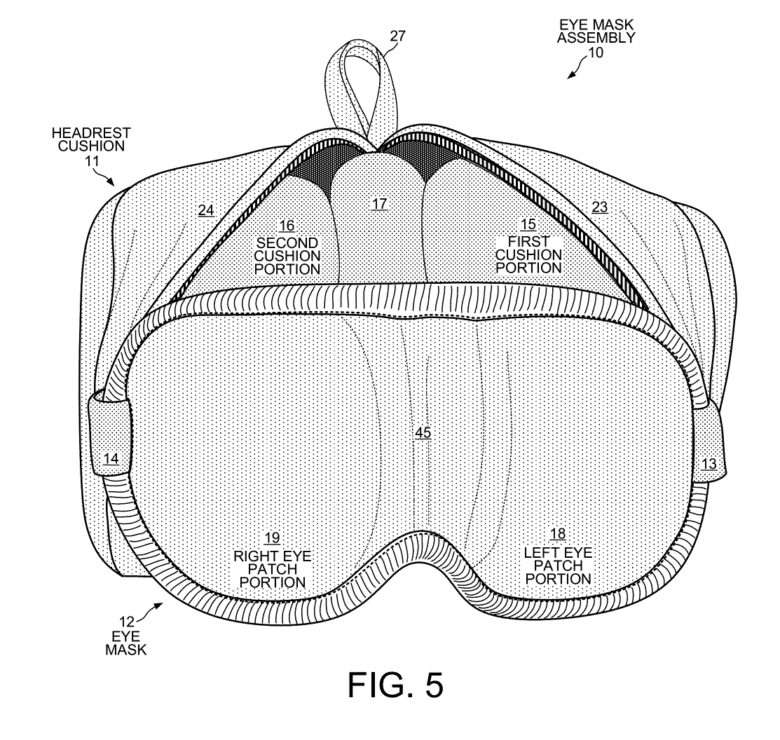

FIG. 5 is a front view of the travel eye mask assembly 10 in which the eye mask 12 has been removed from the inner compartment 25 and is ready to be placed over the head of a passenger. For example, an airline may provide the eye mask assembly 10 as an upgraded complimentary gift to business class passengers. The headrest cushion 11 acts as a case for the eye mask 12.

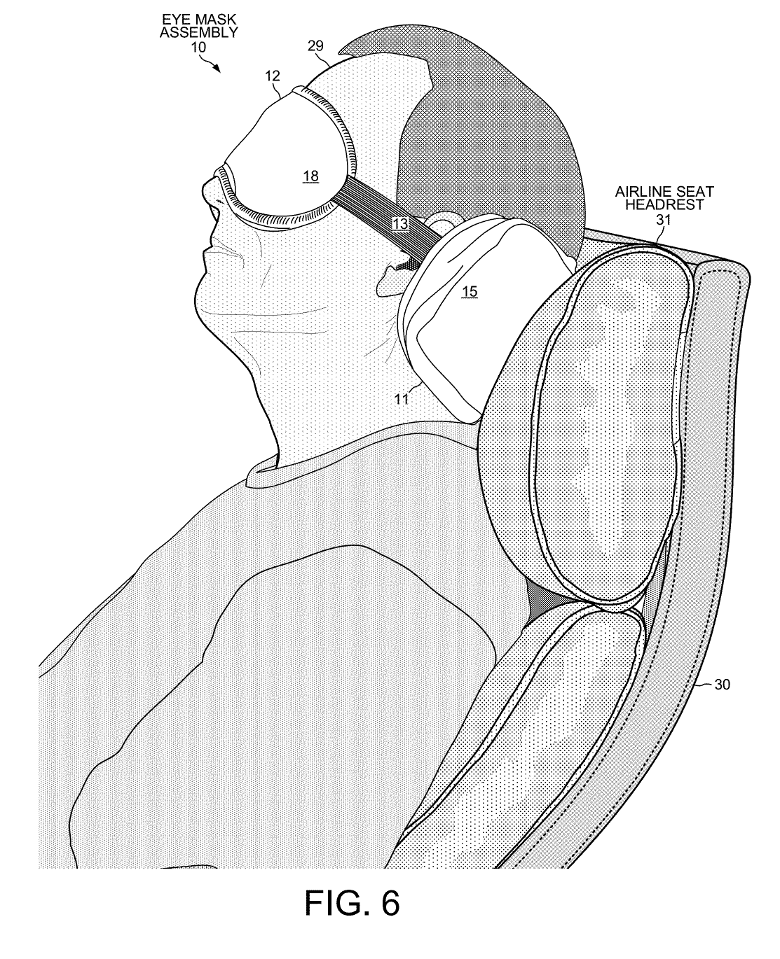

FIG. 6 illustrates a passenger 29 using the travel eye mask assembly 10 in a slightly reclined airplane seat 30. The passenger's upper neck is supported between the first cushion portion 15 and the second cushion portion 16. Supporting the passenger's head from both sides holds the head upright against the headrest 31 and allows the passenger 29 to relax his neck muscles. Because the middle portion 17 of the headrest cushion 11 is less cushioned and thinner than the cushion portions 15-16, the user's head falls farther back into the middle of the headrest cushion 11 and is cradled between the two cushion portions 15-16. Thus, the user's head is prevented from moving to the right or left, and the user's neck does not bend or turn. It is easier for the passenger 29 to sleep because his head is supported and because the eye mask 12 blocks out the light in the cabin. In addition, the straps 13-14 and the ends of the headrest cushion 11, which is between ten and twelve inches long, cover most of the user's ears and block out some of the cabin noise. The elastic straps 13-14 hold the ends of the headrest cushion 11 snugly over the user's ears. In another embodiment, the straps 13-14 include metal strap adjusters that can be used to adjust the lengths of the straps. In yet another embodiment, each of the straps 13-14 can be detached into two segments that can be reattached at different locations to adjust the overall length of the straps. For example, the detachable strap segments can be reattached to one another using hook and loop or tongue and hole connectors on opposite segments.

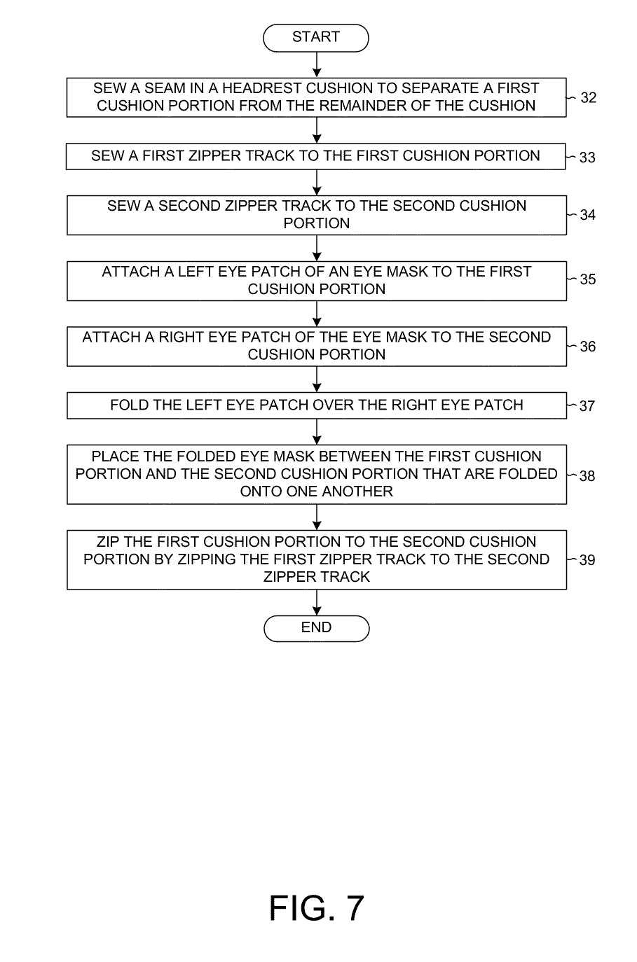

FIG. 7 is a flowchart illustrating steps 32-39 of a method 40 of manufacturing the eye mask and headrest cushion assembly 10. In a first step 32, a first seam 41 is sewn in the headrest cushion 11 to separate the first cushion portion 15 from the remainder of the headrest cushion 11. Then a second seam 42 is sewn in the headrest cushion 11 to separate the second cushion portion 16 from the remainder of the headrest cushion 11. The middle portion 17 of the headrest cushion 11 is formed between the two seams 41-42. The first and second seams 41-42 are shown in FIGS. 2-3. The majority of the padding material or foam pieces is pulled towards the ends of the headrest cushion 11 before the seams 41-42 are sewn so that less cushioning material is present in the middle portion 17. In addition, the first cushion portion 15 is thinner towards the second cushion portion 16 than away from the second cushion portion 16 and vice versa.

In step 33, the first zipper track 21 is sewn onto the first cushion portion 15. A seam 43 is sewn between the fabric strip of the zipper track 21 and the edge of the fabric covering flap 23 that covers the first cushion portion 15.

In step 34, the second zipper track 44 is sewn to the second cushion portion 15. A seam 44 attaches the fabric strip of the zipper track 22 to the edge of the fabric covering flap 24 that covers the second cushion portion 16. The seams 43-44 are shown in FIGS. 1 and 3. The seams 43-44 that attach the zipper tracks 21-22 to the edges of the covering flaps 23-24 are sewn closer to the zipper teeth than to the loose sides 28 of the fabric strips of the zipper tracks.

In step 35, the left eye patch portion 18 of the eye mask 12 is connected to the first cushion portion 15 by the first elastic strap 13. One end of the strap 13 is sewn into an edging seam that surrounds the left eye patch portion 18, and the other end is attached to the first cushion portion 15 inside the covering flap 23.

In step 36, the right eye patch portion 19 of the eye mask 12 is attached to the second cushion portion 16 by the second elastic strap 14.

In step 37, the left eye patch 18 is folded towards the right eye patch 19 as shown in FIG. 2. The two eye patches 18-19 cannot be folded onto one another until the cushion portions 15-16 also begin to be folded towards one another. The crease 45 between the left and right eye patch portions 18-19 is placed on the middle portion 17 of the headrest cushion 11 before the assembly is further folded.

In step 38, the folded eye mask 12 is placed between the first cushion portion 15 and the second cushion portion 16 that are folded over one another.

In step 39, the first cushion portion 15 is joined to the second cushion portion 16 by zipping the first zipper track 21 to the second zipper track 22. Closing the zipper 20 fastens the first covering flap 23 of the first cushion portion 15 to the second covering flap 24 of the second cushion portion 16 and forms the inner compartment 25 in which the eye mask 12 is stored. FIG. 4 shows the eye mask and headrest cushion assembly 10 in a condition ready for delivery to the user after the zipper 20 has been closed in step 39. The travel eye mask assembly 10 is an appropriate travel accessory for individuals who must travel in an upright, seated position for extended periods, such as airline passengers. The assembly 10 allows the user to rest in a slightly reclined posture such that the user's head is prevented from rolling to one side while the user sleeps.

Although certain specific exemplary embodiments are described above in order to illustrate the invention, the invention is not limited to the specific embodiments. Although the headrest cushion 11 is described as being a continuous cushion divided into first, middle and second portions by seams, in other embodiments the first and second cushions are separate structures that are connected by a strap or by fabric, for example. Each of the two cushions has a separate covering flap surrounded by a zipper track. The zipper tracks of the separate cushions can be zipped to one another to enclose the eye mask between the cushions. Accordingly, various modifications, adaptations, and combinations of various features of the described embodiments can be practiced without departing from the scope of the invention as set forth in the claims.

What is claimed is:

1. An eye mask and headrest cushion assembly comprising:
a first cushion portion;
a second cushion portion;
a middle portion that is less cushioned, wherein the second cushion portion is connected to the first cushion portion through the middle portion, wherein the first cushion portion is foldable over the second cushion portion;
an eye mask with a left eye patch portion and a right eye patch portion;
a first strap that connects the first cushion portion to the left eye patch portion; and
a second strap that connects the second cushion portion to the right eye patch portion.

2. The assembly of claim 1, wherein the first cushion portion is foldable onto the second cushion portion when the left eye patch portion is folded into the right eye patch portion and the eye mask is disposed between the first cushion portion and the second cushion portion.

3. The assembly of claim 1, further comprising:
a zipper that includes a first zipper track and a second zipper track, wherein the first zipper track is sewn to the first cushion portion and the second zipper track is sewn to the second cushion portion.

4. The assembly of claim 3, wherein the eye mask fits between the first cushion portion and the second cushion portion when the zipper is zipped closed.

5. The assembly of claim 3, wherein an inner compartment is formed between the first cushion portion and the second cushion portion when the zipper is zipped closed.

6. The assembly of claim 5, wherein the first strap is attached to the first cushion portion inside the inner compartment.

7. The assembly of claim 5, wherein the first cushion portion is thinner near the middle portion than away from the middle portion.

8. The assembly of claim 1, wherein the first cushion portion contains foam.

9. The assembly of claim 1, wherein the first cushion portion, the middle portion and the second cushion portion have a combined length of less than twelve inches.

10. The assembly of claim 1, wherein the first cushion portion, the middle portion and the second cushion portion are sized to cradle an adult person's head at the upper neck.

11. An eye cover device, comprising:
a headrest cushion with a first cushion portion connected to a second cushion portion;
an eye mask with a left eye patch and a right eye patch;
a first strap that connects the first cushion portion to the left eye patch; and
a second strap that connects the second cushion to the right eye patch.

12. The eye cover device of claim 11, wherein the first cushion portion is connected to the second cushion portion through a less cushioned middle portion.

13. The eye cover device of claim 12, further comprising:
a zipper with a first zipper track and a second zipper track, wherein the first zipper track is attached to the first cushion portion and the second zipper track is attached to the second cushion portion.

14. The eye cover device of claim 13, wherein the eye mask is folded by folding the left eye patch over the right eye patch, and wherein the folded eye mask is disposed between the first cushion portion and the second cushion portion when the zipper is zipped closed.

15. The eye cover device of claim 11, wherein the eye mask is disposed in an inner compartment formed between the first cushion portion and the second cushion portion when the zipper is zipped closed.

16. The eye cover device of claim 11, wherein the first cushion portion is thinner towards the second cushion portion than away from the second cushion portion.

17. A method of manufacturing an eye cover device, comprising:
sewing a seam in a headrest cushion to separate a first cushion portion from the remainder of the headrest cushion, wherein the headrest cushion also includes a second cushion portion;
sewing a first zipper track to the first cushion portion;
sewing a second zipper track to the second cushion portion;
attaching a left eye patch of an eye mask to the first cushion portion; and
attaching a right eye patch of the eye mask to the second cushion portion.

18. The method of claim 17, further comprising:
folding the left eye patch towards the right eye patch;
placing the folded eye mask between the first cushion portion and the second cushion portion that are folded onto one another; and
zipping the first cushion portion to the second cushion portion by zipping the first zipper track to the second zipper track.

19. The method of claim 17, wherein the left eye patch is attached to the first cushion portion by an elastic strap.

20. The method of claim 17, further comprising:
folding the eye mask;
placing the folded eye mask between the first cushion portion and the second cushion portion; and
zipping the first zipper track to the second zipper track.
